(19) United States
(12) Patent Application Publication
(10) Pub. No.: US 2021/0403493 A1
(43) Pub. Date: Dec. 30, 2021

(54) USE OF TWO-TAIL LONG-CHAIN ANIONIC SURFACTANTS IN AQUEOUS POLYURETHANE DISPERSIONS

(71) Applicant: Evonik Operations GmbH, Essen (DE)

(72) Inventors: Michael Klostermann, Essen (DE); Kai-Oliver Feldmann, Essen (DE); Jan Marian von Hof, Bochum (DE); Sina Arnold, Bottrop (DE); Marvin Jansen, Essen (DE)

(73) Assignee: Evonik Operations GmbH, Essen (DE)

(21) Appl. No.: 17/321,609

(22) Filed: May 17, 2021

(30) Foreign Application Priority Data
Jun. 24, 2020 (EP) 20181876.2

(51) Int. Cl. C07F 9/11; C08K 5/521; C08K 5/11; C09D 175/08; C07C 69/704

(52) U.S. Cl. CPC C07F 9/11; C08K 5/521; C07C 69/704; C09D 175/08; C08K 5/11

(57) ABSTRACT

The use of two-tail long-chain anionic surfactants as additives in aqueous polymer dispersions for production of porous polymer coatings, preferably for production of porous polyurethane coatings, is described.

### CROSS REFERENCE TO RELATED APPLICATIONS

This application is a 35 U.S.C. § 119 patent application which claims the benefit of European Application No. 20181876.2 filed Jun. 24, 2020, which is incorporated herein by reference in its entirety.

### FIELD

The present invention is in the field of plastics coatings and imitation leathers.

It relates more particularly to the production of porous polymer coatings, especially porous polyurethane coatings, using two-tail long-chain anionic surfactants as additives.

Textiles coated with plastics, for example imitation leathers, generally consist of a textile carrier onto which is laminated a porous polymer layer which has in turn been coated with a top layer or a topcoat.

### BACKGROUND

The porous polymer layer in this context preferably has pores in the micrometre range and is air-permeable and hence breathable, i.e. permeable to water vapor, but water-resistant. The porous polymer layer often comprises porous polyurethane. For environmentally friendly production of PU-based imitation leather, a method based on aqueous polyurethane dispersions, called PUDs, has recently been developed. These generally consist of polyurethane microparticles dispersed in water; the solids content is usually in the range of 30-60% by weight. For production of a porous polyurethane layer, these PUDs are mechanically foamed, coated onto a carrier (layer thicknesses typically between 300-2000 μm) and then dried at elevated temperature. During this drying step, the water present in the PUD system evaporates, which results in formation of a film of the polyurethane particles. In order to further increase the mechanical strength of the film, it is additionally possible to add hydrophilic (poly)isocyanates or carbodiimides to the PUD system during the production process, and these can react with free OH radicals present on the surface of the polyurethane particles during the drying step, thus leading to additional crosslinking of the polyurethane film.

Both the mechanical and the tactile properties of PUD coatings thus produced are determined to a crucial degree by the cell structure of the porous polyurethane film. In addition, the cell structure of the porous polyurethane film affects the air permeability and breathability of the material. Particularly good properties can be achieved here with very fine, homogeneously distributed cells. A customary way of influencing the cell structure during the above-described production process is to add foam stabilizers to the PUD system before or during the mechanical foaming. A first effect of appropriate stabilizers is that sufficient amounts of air can be beaten into the PUD system during the foaming operation. Secondly, the foam stabilizers have a direct effect on the morphology of the air bubbles produced. The stability of the air bubbles is also influenced to a crucial degree by the type of stabilizer. This is important especially during the drying of foamed PUD coatings, since it is possible in this way to prevent drying defects such as cell coarsening or drying cracks.

Various foam stabilizers have already been used in the past in the above-described PUD process. Document US 2015/0284902 A1 or US 2006 0079635 A1, for example, describes the use of ammonium stearate-based foam stabilizers. However, the use of corresponding ammonium stearate-based stabilizers is associated with a number of drawbacks. A significant drawback here is that ammonium stearate has a very high migration capacity in the finished imitation leather. The effect of this is that surfactant molecules accumulate at the surface of the imitation leather with time, which can result in white discoloration at the leather surface. Furthermore, this surfactant migration can result in a greasy film that is perceived as unpleasant on the surface of the imitation leather, especially when corresponding materials come into contact with water.

A further drawback of ammonium stearate is that it forms insoluble lime soaps on contact with hard water. In the case of contact of imitation leather produced on the basis of ammonium stearate with hard water, white efflorescence can thus arise at the imitation leather surface, which is undesirable especially in the case of dark-colored leather.

Yet another drawback of ammonium stearate-based foam stabilizers is that they do permit efficient foaming of aqueous polyurethane dispersions, but often lead to quite a coarse and irregular foam structure. This can have an adverse effect on the optical and tactile properties of the finished imitation leather.

Yet another drawback of ammonium stearate is that the PUD foams produced often have inadequate stability, which can lead to drawbacks in the processing thereof, especially in the drying of the PUD foams at elevated temperatures. A consequence of this would be, for example, that corresponding foams have to be dried relatively gently and slowly, which in turn leads to longer process times in imitation leather production.

As an alternative to ammonium stearate-based foam stabilizers, polyol esters and polyol ethers were identified in the past as effective foam additives for aqueous polyurethane dispersions. The structures are described, for example, in documents EP 3487945 A1 and WO2019042696A1. Compared to ammonium stearate, polyol esters and polyol ethers have the major advantage that they migrate only slightly, if at all, in the finished imitation leather and hence do not lead to unwanted surface discoloration. Moreover, polyol esters and polyol ethers are not sensitive to hard water.

A further advantage of polyol esters and polyol ethers over ammonium stearate-based foam stabilizers is additionally that they often lead to a distinctly finer and more homogeneous foam structure, which has advantageous effects on the properties of imitation leather materials produced with these substances. Polyol esters and polyol ethers often also lead to much more stable PUD foams, which in turn brings process-related advantages in imitation leather production.

In spite of these advantages, polyol esters and polyol ethers are also not entirely free of potential drawbacks. A potential drawback here is that the foam-stabilizing effect of these compound classes can be impaired under some circumstances by the presence of further cosurfactants present in the PUD system. Especially in the production of aqueous polyurethane dispersions, however, the use of cosurfactants is not unusual. Cosurfactants are used in this context for improved dispersion of polyurethane prepolymers in water and generally remain in the final product. During the mechanical foaming of aqueous polyurethane dispersions containing polyol esters or polyol ethers as foam additives, corresponding cosurfactants can have adverse effects on the foaming characteristics of the system under some circumstances. As a result, under some circumstances, it is often possible for only little air, if any at all, to be beaten into the system; this could be detrimental to the resultant foam structure. Cosurfactants can also have an adverse effect on the stability of the foams produced, which can result in foam ageing during the processing of the foamed PUD system, which in turn leads to faults and defects in the foam coatings produced.

A further potential drawback is that PUD systems containing polyol esters or polyol ethers as foam additives often require very high shear energies for efficient foaming. This in turn can entail limitations and process-related drawbacks under some circumstances.

### SUMMARY

The problem addressed by the present invention was therefore that of providing additives for production of PUD-based foam systems and foam coatings that enable efficient foaming of PUD systems and do not have the drawbacks detailed in the art. It has been found that, surprisingly, two-tail long-chain anionic surfactants enable the solution of the stated problem.

### DETAILED DESCRIPTION

The present invention therefore provides for the use of two-tail long-chain anionic surfactants as additives, preferably as foam additives, in aqueous polymer dispersions, preferably aqueous polyurethane dispersions, for production of porous polymer coatings, preferably for production of porous polyurethane coatings.

The inventive use of two-tail long-chain anionic surfactants surprisingly has various advantages here.

One advantage is that two-tail long-chain anionic surfactants enable particularly efficient foaming of aqueous PUD systems. The foams thus produced are notable here for an exceptionally fine pore structure with particularly homogeneous cell distribution, which in turn has a very advantageous effect on the mechanical and tactile properties of the porous polymer coatings which are produced on the basis of these foams. In addition, it is possible in this way to improve the air permeability or breathability of the coating.

A further advantage is that two-tail long-chain anionic surfactants, even at relatively low shear rates, enable efficient foaming of PUD systems, which leads to fewer limitations and broader processibility during imitation leather production.

Yet another advantage is that two-tail long-chain anionic surfactants enable the production of particularly stable foams. This firstly has an advantageous effect on their processibility. Secondly, the elevated foam stability has the advantage that, during the drying of corresponding foams, drying defects such as cell coarsening or drying cracks can be avoided. Furthermore, the improved foam stability enables quicker drying of the foams, which offers processing advantages both from an environmental and from an economic point of view.

Yet another advantage is that the efficacy of two-tail long-chain anionic surfactants is barely impaired, if at all, by cosurfactants present in the PUD system. Thus, the surfactant formulations according to the invention, even in the case of cosurfactant-containing PUD systems, enable efficient foaming of the system, and the formation of fine and homogeneous foams that are simultaneously extremely stable.

Yet another advantage is that the two-tail long-chain anionic surfactants according to the invention, in the finished imitation leather, have barely any migration capacity, if any, and thus do not lead to unwanted surface discoloration or efflorescence. Furthermore, the surfactants according to the invention are barely sensitive to hard water, if at all.

The expression “two-tail long-chain anionic surfactant” throughout the present invention encompasses surfactants having an anionic hydrophilic head group and two identical or different long-chain hydrophobic hydrocarbyl radicals. What is meant by “long-chain” in this context is that the hydrophobic hydrocarbyl radicals consist of at least 12, preferably at least 14, carbon atoms, more preferably of at least 16 carbon atoms.

The term “cosurfactant” throughout the present invention encompasses additional surfactants that may be present in the polymer dispersion alongside the two-tail long-chain anionic surfactants according to the invention. These especially include surfactants that are used during the production of the polymer dispersion. For example, polyurethane dispersions are often produced by synthesis of a PU prepolymer which, in a second step, is dispersed in water and then reacted with a chain extender. For improved dispersion of the prepolymer in water, it is possible here to use cosurfactants. In the context of the present invention, the cosurfactants are preferably anionic cosurfactants.

The invention is described further and by way of example hereinafter, without any intention that the invention be restricted to these illustrative embodiments. Where ranges, general formulae or compound classes are specified below, these are intended to include not only the corresponding ranges or groups of compounds which are explicitly mentioned but also all subranges and subgroups of compounds which can be obtained by removing individual values (ranges) or compounds. Where documents are cited in the context of the present description, the entire content thereof, particularly with regard to the subject matter that forms the context in which the document has been cited, is intended to form part of the disclosure content of the present invention. Unless otherwise stated, percentages are in percent by weight. Where parameters that have been determined by measurement are given hereinbelow, the measurements have been carried out at a temperature of 25° C. and a pressure of 101 325 Pa, unless otherwise stated. Where chemical (empirical) formulae are used in the present invention, the specified indices can be not only absolute numbers but also average values. For polymeric compounds, the indices preferably represent average values. Structural and empirical formulae presented in the present invention are representative of all isomers that are possible by differing arrangement of the repeating units.

In the context of the present invention, preference is given to those two-tail long-chain anionic surfactants that consist of an anionic head group and long-chain hydrophobic radicals. The hydrophobic radicals here may independently be identical or different monovalent aliphatic or aromatic, saturated or unsaturated hydrocarbyl radicals having 12 to 40 carbon atoms, preferably 14 to 30, more preferably having 16 to 24 carbon atoms. The anionic head group is preferably based here either on organic carboxylates or anionic sulfur or phosphorus compounds. More preferably, the anionic head group has structural units selected from the group of the carboxylates, the phosphates, the phosphonates, the phosphinates, the sulfates and the sulfonates. In the context of the present invention, preference is given especially to two-tail long-chain anionic surfactants conforming to the general formula (I)

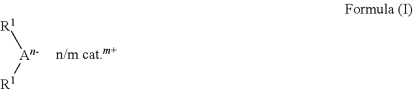

where the R1 radicals are independently identical or different monovalent saturated or unsaturated, aliphatic or aromatic hydrocarbyl radicals having 12 to 40 carbon atoms, preferably 14 to 30, more preferably having 16 to 24 carbon atoms, and where An− is the anionic head group of the surfactant that bears n negative charges, where n=1-3, more preferably 1-2, even more preferably 1, and where cat.^{m+} is the cationic counterion that bears m positive charges, where m=1-10, preferably 1-5, more preferably 1-2, even more preferably 1.

The R¹ radicals can be attached here to the anionic head group via many kinds of linkage motifs, for example via, but not limited to, ester, amide, ether, carbonate or silicone bonds. In addition, the R¹ radicals can be attached to the anionic head group via polyoxyalkylene bridges, preferably via polyoxyethylene and polyoxypropylene bridges. Particular preference is given in this connection to polyoxyalkylene, preferably polyoxyethylene and polyoxypropylene, bridges having not more than 10, preferably not more than 7, more preferably not more than 5, even more preferably not more than 3, alkoxy units. In this case, corresponding polyoxyalkylene, preferably polyoxyethylene and polyoxypropylene, bridges should be considered part of the anionic head group. Combinations of the linking and bridging motifs mentioned are likewise preferred in the context of the present invention.

In the context of the present invention, it is preferable when the anionic head group Aⁿ⁻ has structural features of organic carboxylates or anionic sulfur or phosphorus compounds. More preferably, the anionic head group here has structural units selected from the group of the carboxylates, the phosphates, the phosphonates, the phosphinates, the sulfates and the sulfonates.

Preferred R¹ radicals derive from long-chain alcohols, carboxylic acids or alkylamines having at least 12 to 40 carbon atoms, preferably 14 to 30, more preferably having 16 to 24 carbon atoms, and mixtures of these substances.

If the R¹ radical derives from long-chain alcohols, these are preferably lauryl alcohol (1-dodecanol), myristyl alcohol (1-tetradecanol), cetyl alcohol (1-hexadecanol), margaryl alcohol (1-heptadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), melissyl alcohol (1-triacontanol), palmitoleyl alcohol (cis-9-hexadecen-1-ol), oleyl alcohol (cis-9-octadecen-1-ol) and/or elaidyl alcohol (trans-9-octadecen-1-ol) and mixtures of these substances, particular preference being given to palmitoleyl alcohol and stearyl alcohol and to mixtures of these two substances. Preference is given here in accordance with the invention both to the pure alcohols and to their technical grade qualities having chain length distributions, or containing mixtures of fatty acids of different chain length.

If the R¹ radical derives from long-chain carboxylic acids, these are preferably selected from lauric acid (dodecanoic acid), myristic acid (tetradecanoic acid), palmitic acid (hexadecanoic acid), stearic acid (octadecanoic acid), arachic acid (eicosanoic acid), behenic acid (docosanoic acid), lignoceric acid (tetracosanoic acid), palmitoleic acid ((Z)-9-hexadecenoic acid), oleic acid ((Z)-9-hexadecenoic acid), elaidic acid ((E)-9-octadecenoic acid), cis-vaccenic acid ((Z)-11-octadecenoic acid), linoleic acid ((9Z,12Z)-9,12-octadecadienoic acid), alpha-linolenic acid ((9Z,12Z,15Z)-9,12,15-octadecatrienoic acid), gamma-linolenic acid ((6Z,9Z,12Z)-6,9,12-octadecatrienoic acid), di-homo-gamma-linolenic acid ((8Z,11Z,14Z)-8,11,14-eicosatrienoic acid), arachidonic acid ((5Z,8Z,11Z,14Z)-5,8,11,14-eicosatetraenoic acid), erucic acid ((Z)-13-docosenoic acid), nervonic acid ((Z)-15-tetracosenoic acid), ricinoleic acid, hydroxystearic acid and/or undecenyloic acid, and also mixtures thereof, for example rapeseed oil acid, soya fatty acid, sunflower fatty acid, peanut fatty acid and tall oil fatty acid. Very particular preference is given to palmitic acid and stearic acid, and especially the mixtures of these substances.

Preference is given here in accordance with the invention both to the pure carboxylic acids and to their technical grade qualities having chain length distributions, or containing mixtures of fatty acids of different chain length.

If the R¹ radical derives from long-chain amines, these are especially laurylamine (1-dodecylamine), myristylamine (1-tetradecylamine), cetylamine (1-hexadecylamine), margarylamine (1-heptadecylamine), stearylamine (1-octadecylamine), arachidylamine (1-eicosylamine), behenylamine (1-docosylamine), lignocerylamine (1-tetracosylamine), cerylamine (1-hexacosylamine), montanylamine (1-octacosylamine), melissylamine (1-triacontylamine), palmitoleylamine (cis-9-hexadecenylamine), oleylamine (cis-9-octadecenylamine) and/or elaidylamine (trans-9-octadecenylamine) and mixtures, particular preference being given to palmitoleylamine and stearylamine and to mixtures of these two substances. Preference is given here in accordance with the invention both to the pure amines and to their technical grade qualities having chain length distributions, or containing mixtures of fatty acids of different chain length.

Sources of the above-described long-chain alcohols, amines and carboxylic acids may be vegetable or animal fats, oils or waxes. For example, it is possible to use: pork lard, beef tallow, goose fat, duck fat, chicken fat, horse fat, whale oil, fish oil, palm oil, olive oil, avocado oil, seed kernel oils, coconut oil, palm kernel oil, cocoa butter, cottonseed oil, pumpkinseed oil, maize kernel oil, sunflower oil, wheatgerm oil, grapeseed oil, sesame oil, linseed oil, soybean oil, peanut oil, lupin oil, rapeseed oil, mustard oil, castor oil, jatropha oil, walnut oil, jojoba oil, lecithin, for example based on soya, rapeseed or sunflowers, bone oil, neatsfoot oil, borage oil, lanolin, emu oil, deer tallow, marmot oil, mink oil, safflower oil, hemp oil, pumpkin oil, evening primrose oil, tall oil, and also carnauba wax, beeswax, candelilla wax, ouricury wax, sugarcane wax, retamo wax, caranday wax, raffia wax, esparto wax, alfalfa wax, bamboo wax, hemp wax, Douglas fir wax, cork wax, sisal wax, flax wax, cotton wax, dammar wax, tea wax, coffee wax, rice wax, oleander wax and/or wool wax.

In the context of the present invention, preference is further given to those two-tail long-chain anionic surfactants conforming to the general formula (II)

R²-Aⁿ⁻n/m cat.^{m+} Formula (II)

where R² is a branched hydrocarbyl radical that in turn consists of two identical or different monovalent aliphatic or aromatic, saturated or unsaturated hydrocarbyl radicals each having 12 to 40 carbon atoms, preferably 14 to 30, more preferably having 16 to 24 carbon atoms, and Aⁿ⁻ and cat.^{m+} and the attachment of the R² radical to the hydrophilic head group are as described above.

In the context of the present invention, the R² radicals preferably derive from branched primary and/or secondary alcohols. Preference is given here especially to Guerbet alcohols, i.e. branched alcohols formed by Guerbet condensation, and to branched secondary alcohols formed by paraffin oxidation by the Bashkirov method. Preference is also given in this connection to alkoxylates, preferably ethoxylates and propoxylates, of the aforementioned alcohols, preferably having not more than 10, preferably having not more than 7, more preferably having not more than 5, even more preferably having not more than 3, alkoxy units.

It is especially preferable in this connection when the two-tail long-chain anionic surfactants conform to the general formula (III) or (IV):

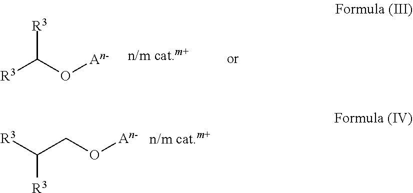

where the R³ radicals are independently identical or different monovalent aliphatic or aromatic, saturated or unsaturated hydrocarbyl radicals having 12 to 40 carbon atoms, preferably 14 to 30, more preferably having 16 to 24 carbon atoms, and Aⁿ⁻ and cat.^{m+} are as described above.

In the context of the present invention, preference is given especially to those two-tail long-chain anionic surfactants that are selected from the groups of the dialkyl sulfosuccinates, the dialkyl phosphates, the dialkyl sulfosuccinamates, and the dialkyl esters of trifunctional or higher-functionality carboxylic acids. Particular preference is given in this context to dipalmityl sulfosuccinates, distearyl sulfosuccinates, stearyl palmityl sulfosuccinates, dipalmityl phosphates, distearyl phosphates, stearyl palmityl phosphates, dipalmityl sulfosuccinamates, distearyl sulfosuccinamates, stearyl palmityl sulfosuccinamates, dipalmityl esters of trifunctional or higher-functionality carboxylic acids, distearyl esters of trifunctional or higher-functionality carboxylic acids, stearyl palmityl esters of trifunctional or higher-functionality carboxylic acids and mixtures of these substances. In the context of the present invention, preference is further given to those two-tail long-chain anionic surfactants that are selected from the group of the monoalkyl sulfosuccinates, monoesters of phosphoric acid, sulfuric acid and dibasic or higher polybasic carboxylic acids with long-chain branched alcohols, as described above.

The long-chain dialkyl sulfosuccinates or monoalkyl sulfosuccinates that are based on branched long-chain alcohols and are preferred in the context of the present invention are obtainable synthetically, for example, by reacting maleic anhydride with corresponding alcohols, as described above, followed by sulfonation with sodium hydrogensulfite.

The dialkyl sulfosuccinamates preferred in the context of the present invention are obtainable synthetically, for example, by reacting maleic anhydride with long-chain amines, as described above, followed by sulfonation with sodium hydrogensulfite.

The long-chain dialkyl phosphates, and monophosphates of branched long-chain alcohols, that are preferred in the context of the present invention are obtainable, for example, by reacting phosphoric anhydride (P₄O₁₀) with corresponding alcohols, as described above, followed by neutralization of the partial phosphoric ester formed.

The dialkyl esters of trifunctional or higher-functionality carboxylic acids, and monoesters of difunctional or higher-functionality carboxylic acids with branched long-chain alcohols, that are preferred in the context of the present invention are obtainable synthetically, for example, by condensing corresponding carboxylic acids with corresponding alcohols, as described above, followed by neutralization of the partial esters formed.

The monosulfate esters of branched long-chain alcohols that are preferred in the context of the present invention are obtainable, for example, by reacting sulfur trioxide with corresponding alcohols, as described above, followed by neutralization of the partial sulfuric ester formed.

The term “neutralization” over the entire scope of the present invention also covers partial neutralization. For neutralization, including partial neutralization, it is possible to use customary bases. These include the water-soluble metal hydroxides, for example barium hydroxide, strontium hydroxide, calcium hydroxide and preferably the hydroxides of the alkali metals that dissociate into free metal and hydroxide ions in aqueous solutions, especially NaOH and KOH. These also include the anhydro bases which react with water to form hydroxide ions, for example barium oxide, strontium oxide, calcium oxide, lithium oxide, silver oxide and ammonia. As well as these aforementioned alkalis, solid substances usable as bases are also those which likewise give an alkaline reaction on dissolution in water without having HO− (in the solid compound); examples of these include amines such as mono-, di- and trialkylamines, which may also be functionalized alkyl radicals as, for example, in the case of amide amines, mono-, di- and trialkanolamines or mono-, di- and triaminoalkylamines, and, for example, the salts of weak acids, such as potassium carbonate, sodium carbonate, trisodium phosphate, etc. In addition, it is also possible to use higher-functionality amines, for example ethylenediamine, diethylenetriamine or triethylenetetramine, for neutralization.

In the context of the present invention, it is further preferable when, in formula (I)-(IV), the cation cat.^{m+} is a metal cation, more preferably a lithium, sodium, potassium, calcium, strontium, barium or silver cation, very particular preference being given to sodium or potassium cations. It is further preferable when the cation is an ammonium cation of a protonated amine compound, particular preference being given to ammonium ions based on ammonia, amines such as mono-, di- and trialkylamines, where the alkyl radicals may also be functionalized as, for example, in amide amines, mono-, di- and trialkanolamines or mono-, di- and triaminoalkylamines, and higher-functionality amines, for example ethylenediamine, diethylenetriamine or triethylenetetramine.

As described above, it is possible to obtain the two-tail long-chain anionic surfactants in the context of the present invention by neutralization of partial esters of higher polybasic acids, for example long-chain partial phosphoric esters. Since, in the context of the present invention, the term “neutralization” also covers partial neutralization, the cation K in this case may also be a hydrogen atom bonded to the head group or a dissociated hydronium ion.

As already described, the present invention envisages the use of long-chain two-tail anionic surfactants as described above as additives in aqueous polymer dispersions, preferably in aqueous polyurethane dispersions. The polymer dispersions here are preferably selected from the group of aqueous polystyrene dispersions, polybutadiene dispersions, poly(meth)acrylate dispersions, polyvinyl ester dispersions and/or polyurethane dispersions. The solids content of these dispersions is preferably in the range of 20-70% by weight, more preferably in the range of 25-65% by weight. Particular preference is given in accordance with the invention to the use of long-chain two-tail anionic surfactants as additives in aqueous polyurethane dispersions. Especially preferable here are polyurethane dispersions based on polyester polyols, polyesteramide polyols, polycarbonate polyols, polyacetal polyols and/or polyether polyols.

In the context of the present invention, it is preferable when the concentration of two-tail long-chain anionic surfactants, based on the total weight of the aqueous polymer dispersion, is in the range of 0.1-20% by weight, more preferably in the range of 0.2-15% by weight, especially preferably in the range of 0.5-10% by weight.

Preference is given to using the two-tail long-chain anionic surfactants in aqueous polymer dispersions as foaming aids or foam stabilizers for foaming of the dispersions, i.e. as foam additives. In addition, however, they may also be used as drying auxiliaries, levelling additives, wetting agents and rheology additives, which likewise corresponds to preferred embodiments of the present invention.

As well as the two-tail long-chain anionic surfactants according to the invention, the aqueous polymer dispersions may also comprise further additions/formulation components such as color pigments, fillers, flatting agents, stabilizers such as hydrolysis or UV stabilizers, antioxidants, absorbers, crosslinkers, levelling additives, thickeners and further cosurfactants.

The two-tail long-chain anionic surfactants can be added to the aqueous dispersion either in pure or blended form in a suitable solvent. Preferred solvents in this connection are selected from water, propylene glycol, dipropylene glycol, polypropylene glycol, butyldiglycol, butyltriglycol, ethylene glycol, diethylene glycol, polyethylene glycol, polyalkylene glycols based on EO, PO, BO and/or SO, alcohol alkoxylates based on EO, PO, BO and/or SO, and mixtures of these substances, very particular preference being given to aqueous dilutions or blends. Blends or dilutions of the two-tail long-chain anionic surfactants include at least 5% by weight, more preferably at least 10% by weight, even more preferably at least 15% by weight, of the two-tail long-chain anionic surfactants.

In the case of aqueous dilutions or blends of the two-tail long-chain anionic surfactants according to the invention, it may be advantageous when hydrotropic compounds are added to the blend to improve the formulation properties (viscosity, homogeneity, etc.). Hydrotropic compounds here are water-soluble organic compounds consisting of a hydrophilic part and a hydrophobic part, but are too low in molecular weight to have surfactant properties. They lead to an improvement in the solubility or in the solubility properties of organic, especially hydrophobic organic, substances in aqueous formulations. The term “hydrotropic compounds” is known to those skilled in the art. Preferred hydrotropic compounds in the context of the present invention are alkali metal and ammonium toluenesulfonates, alkali metal and ammonium xylenesulfonates, alkali metal and ammonium naphthalenesulfonates, alkali metal and ammonium cumenesulfonates, and phenol alkoxylates, especially phenol ethoxylates, having up to 6 alkoxylate units.

It may also be advantageous for the two-tail long-chain anionic surfactants to be used not in pure form but in combination with further cosurfactants as additives in aqueous polymer dispersions, preferably in aqueous polyurethane dispersions. These may be used, for example, for improved system compatibility or, in the case of pre-formulated surfactant mixtures, for improved formulation properties. Cosurfactants preferred in accordance with the invention in this context are, for example, free fatty alcohols, fatty acid amides, ethylene oxide-propylene oxide block copolymers, betaines, for example amidopropyl betaines, amine oxides, quaternary ammonium surfactant, amphoacetates, ammonium and/or alkali metal salts of fatty acid, alkyl sulfates, alkyl ether sulfates, alkylsulfonates, alkylbenzenesulfonates, alkyl phosphates, alkyl sulfosuccinates, alkyl sulfosuccinamates, alkyl sarcosinates and mixtures of these substances, very particular preference being given to free fatty alcohols, preferably having 12 to 40, more preferably having 14-30, even more preferably having 16-24, carbon atoms, and alkyl sulfates having 12 to 40, more preferably having 14-30, even more preferably having 16-24, carbon atoms, and mixtures of these substances. In addition, the cosurfactant may comprise silicone-based surfactants, for example trisiloxane surfactants or polyether siloxanes. In the case of ammonium and/or alkali metal salts of fatty acids, it is preferable when they contain less than 25% by weight of stearate salts, and are especially free of stearate salts.

In the case of combinations of the two-tail long-chain anionic surfactants according to the invention with further cosurfactants, as described above, it is especially preferred when these combinations include between 1% and 60% by weight, preferably between 2% and 50% by weight, more preferably between 3% and 40% by weight, even more preferably between 5% and 30% by weight, of cosurfactant, based on the combination of two-tail long-chain anionic surfactants according to the invention and cosurfactant.

Since, as described above, the inventive use of two-tail long-chain anionic surfactants leads to a distinct improvement in porous polymer coatings produced from aqueous polymer dispersions, the present invention likewise provides aqueous polymer dispersions comprising at least one of the two-tail long-chain anionic surfactants according to the invention, as described in detail above.

The present invention still further provides porous polymer layers which have been produced from aqueous polymer dispersions, obtained with the inventive use of two-tail long-chain anionic surfactants, as described in detail above.

Preferably, the porous polymer coatings according to the invention can be produced by a process comprising the steps of
a) providing a mixture comprising at least one aqueous polymer dispersion, at least one two-tail long-chain anionic surfactant according to the invention, and optionally further additives,
b) foaming the mixture to give a foam,
c) optionally adding at least one thickener to adjust the viscosity of the wet foam,
d) applying a coating of the foamed polymer dispersion to a suitable carrier,
e) drying/curing the coating.

The porous polymer coatings have pores, preferably in the micrometre range, more preferably having an average cell size of less than 350 μm, further preferably less than 200 μm, especially preferably less than 150 μm, most preferably less than 100 μm. The average cell size can preferably be determined by microscopy, preferably by electron microscopy. For this purpose, a cross section of the porous polymer coating is viewed by means of a microscope with sufficient magnification, and the size of at least 25 cells is ascertained. The average cell size is then found as the arithmetic average of the cells or cell sizes viewed.

With a view to preferred configurations, especially with a view to the long-chain two-tail anionic surfactants and polymer dispersions that are usable with preference in the process, reference is made to the preceding description and also to the aforementioned preferred embodiments, especially as detailed in the claims.

It is made clear that the process steps of the process according to the invention as set out above are not subject to any fixed sequence in time. For example, process step c) can be executed at an early stage, at the same time as process step a).

It is a preferred embodiment of the present invention when, in process step b), the aqueous polymer dispersion is foamed by the application of high shear forces. The foaming can be effected here with the aid of shear units familiar to the person skilled in the art, for example Dispermats, dissolvers, Hansa mixers or Oakes mixers.

In addition, it is preferable when the wet foam produced at the end of process step c) has a viscosity of at least 5, preferably of at least 10, more preferably of at least 15 and even more preferably of at least 20 Pa·s, but of not more than 500 Pa·s, preferably of not more than 300 Pa·s, more preferably of not more than 200 Pa·s and even more preferably of not more than 100 Pa·s. The viscosity of the foam can be determined here preferably with the aid of a Brookfield viscometer, LVTD model, equipped with an LV-4 spindle. Corresponding test methods for determination of the wet foam viscosity are known to those skilled in the art.

In a preferred embodiment of the present invention, in process step b), the foam has a maximum level of homogeneity and cell fineness. The person skilled in the art is able to verify this if desired in the customary manner by simple direct visual inspection by the naked eye or with visual aids, for example magnifying glasses, microscopes, using their experience. “Cell fineness” refers to the cell size. The smaller the average cell size, the finer the foam cells. If desired, cell fineness can be determined, for example, with a light microscope or with a scanning electron microscope. “Homogeneous” means cell size distribution. A homogeneous foam has a very narrow cell size distribution, such that all cells are roughly of the same size. It would again be possible to quantify this with a light microscope or with a scanning electron microscope.

As already described above, additional thickeners can be added to the system to adjust the wet foam viscosity.

Preferably, the thickeners which can be used advantageously in the context of the invention are selected here from the class of the associative thickeners. Associative thickeners here are substances which lead to a thickening effect through association at the surfaces of the particles present in the polymer dispersions. The term is known to those skilled in the art. Preferred associative thickeners are selected here from polyurethane thickeners, hydrophobically modified polyacrylate thickeners, hydrophobically modified polyether thickeners and hydrophobically modified cellulose ethers. Very particular preference is given to polyurethane thickeners. In addition, it is preferable in the context of the present invention when the concentration of the thickeners based on the overall composition of the dispersion is in the range of 0.01-10% by weight, more preferably in the range of 0.05-5% by weight, most preferably in the range of 0.1-3% by weight.

In the context of the present invention, it is additionally preferable when, in process step d), coatings of the foamed polymer dispersion with a layer thickness of 10-10 000 μm, preferably of 50-5000 μm, more preferably of 75-3000 μm, even more preferably of 100-2500 μm, are produced. Coatings of the foamed polymer dispersion can be produced by methods familiar to the person skilled in the art, for example knife coating. It is possible here to use either direct or indirect coating processes (called transfer coating).

It is also preferable in the context of the present invention when, in process step e), the drying of the foamed and coated polymer dispersion is effected at elevated temperatures. Preference is given here in accordance with the invention to drying temperatures of min. 50° C., preferably of 60° C., more preferably of at least 70° C. In addition, it is possible to dry the foamed and coated polymer dispersions in multiple stages at different temperatures, in order to avoid the occurrence of drying defects. Corresponding drying techniques are widespread in industry and are known to those skilled in the art.

As already described, process steps c)-e) can be effected with the aid of widely practised methods known to those skilled in the art. An overview of these is given, for example, in “Coated and laminated Textiles” (Walter Fung, CR-Press, 2002).

In the context of the present invention, preference is given especially to those porous polymer coatings comprising two-tail long-chain anionic surfactants and having an average cell size less than 350 μm, preferably less than 200 μm, especially preferably less than 150 μm, most preferably less than 100 μm. The average cell size can preferably be determined by microscopy, preferably by electron microscopy. For this purpose, a cross section of the porous polymer coating is viewed by means of a microscope with sufficient magnification and the size of at least 25 cells is ascertained. In order to obtain sufficient statistics for this evaluation method, the magnification of the microscope chosen should preferably be such that at least 10×10 cells are present in the observation field. The average cell size is then calculated as the arithmetic average of the cells or cell sizes viewed. This determination of cell size by means of microscopy is familiar to those skilled in the art.

The porous polymer layers (or polymer coatings) according to the invention, comprising at least one of the two-tail long-chain anionic surfactants according to the invention and optionally further additives, may be used, for example, in the textile industry, for example for imitation leather materials, in the construction industry, in the electronics industry, in the sports industry or in the automobile industry. For instance, on the basis of the porous polymer coatings according to the invention, it is possible to produce everyday articles such as shoes, insoles, bags, suitcases, small cases, clothing, automobile parts, preferably seat covers, coverings of door parts, dashboard parts, steering wheels and/or handles, and gearshift gaiters, fitout articles such as desk pads, cushions or seating furniture, gap fillers in electronic devices, cushioning and damping materials in medical applications, or adhesive tapes.

### EXAMPLES

Substances:

SYNTEGRA® YS:3000: MDI (methyl diphenyl diisocyanate)-based polyurethane dispersion from DOW. As a result of the process for preparing it, the product contains 1-3% by weight of the anionic cosurfactant sodium dodecylbenzenesulfonate (CAS: 25155-30-0).

IMPRANIL® DLU: aliphatic polycarbonate ester-polyether-polyurethane dispersion from Covestro

REGEL® WX 151: aqueous polyurethane dispersion from Cromogenia

CROMELASTIC® PC 287 PRG: aqueous polyurethane dispersion from Cromogenia

STOKAL® STA: ammonium stearate (about 30% in H₂O) from Bozetto

STOKAL® SR: tallow fat-based sodium sulfosuccinamate (about 35% in H₂O) from Bozetto

Sodium dodecylbenzenesulfonate (LAS; CAS: 25155-30-0) was sourced from Sigma Aldrich. This is a standard cosurfactant used for production of aqueous polyurethane dispersions.

ECO Pigment Black: aqueous pigment dispersion (black) from Cromogenia.

TEGOWET® 250: polyethersiloxane-based levelling additive from Evonik Industries AG

ORTEGOL® PV 301: polyurethane-based associative thickener from Evonik Industries AG.

REGEL® TH 27: isocyanate-based levelling additive from Cromogenia

Viscosity Measurements:

All viscosity measurements were conducted with a Brookfield viscometer, LVTD, equipped with an LV-4 spindle, at a constant rotation speed of 12 rpm. For the viscosity measurements, the samples were transferred into a 100 ml jar into which the measurement spindle was immersed. The display of a constant viscometer measurement was always awaited.

Method for Determining the Acid Number:

Suitable methods for determining the acid number are particularly those according to DGF C-V 2, DIN EN ISO 2114, Ph. Eur. 2.5.1, ISO 3682 and ASTM D 974.

### Example 1: Synthesis of Stearyl Phosphate

Stearyl alcohol (≥95%, 178.7 g, 0.661 mol) was heated to 70° C. while stirring and with introduction of N₂, and then P₄O₁₀ (21.31 g, 0.0751 mol) was added in small portions, such that the temperature of the reaction mixture did not rise above 80° C. After the addition of the P₄O₁₀ had ended, the reaction mixture was stirred at 80° C. with introduction of N₂ for 3 h, until the acid number was constant. After pouring out and cooling, a colorless solid having an acid number of 127 mg KOH/g was obtained.

### Example 2: Synthesis of Stearyl Citrate

Stearyl alcohol (≥95%, 275.2 g, 1.02 mol, 2.1 eq.) was heated to 70° C. while stirring and with introduction of N₂, and then citric acid (anhydrous, 93.10 g, 0.485 mol, 1.0 eq.) was added. The reaction mixture was heated to 140° C. and stirred with introduction of N₂ at a pressure of 150 mbar for 3 h until the acid number of 62 mg KOH/g had been attained. The mixture was filtered at 2 bar through a pressure filter press (1 μm) at a temperature of 120° C. After cooling, a colorless solid having an acid number of 57 mg KOH/g was obtained.

### Example 3: Comparative Example

As well as the surfactants according to the invention, a comparative surfactant based on polyglycerol-3 stearate was also used, which was prepared by reacting 103.3 g of polyglycerol—OHN=1124 mg KOH/g, Mw=240 g/mol—with 155.0 g of technical grade stearic acid.

### Example 4: Blending of the Surfactants

The surfactants according to the invention from Examples 1 and 2 and the polyglycerol-based comparative surfactant from Example 3 were blended according to the compositions detailed in Table 1 and then homogenized at 80° C. The inventive surfactant formulations 1 and 2 were neutralized to pH=7 with KOH after blending. Comparative surfactant 3 already had a pH of 7 after blending and was not neutralized.

TABLE 1
Composition of surfactant blends used hereinafter:

| | Surfactant 1 | | Surfactant 2 | | Surfactant 3 | |
|---|---|---|---|---|---|---|
| Stearyl phosphate | 20.0 | g | | | — | |
| (from Example 1) | | | | | | |
| Stearyl citrate | | | 20.0 | g | | |
| (from Example 2) | | | | | | |
| Polyglycerol-3 stearate | | | | | 20.0 | g |
| (comparative example) | | | | | | |
| Stearyl alcohol | 4 | g | 4 | g | 4 | g |
| Water | 76.0 | g | 76.0 | g | 76.0 | g |

### Example 5: Foaming Tests

To test the efficacy of the additive combination according to the invention, a series of foaming experiments was conducted. For this purpose, in a first step, the IMPRANIL® DLU polyurethane dispersion from Covestro was used. The foam stabilizers used were the inventive surfactant formulations 1 and 2 (see table 1) and a combination of the two surfactants Stokal STA (ammonium stearate) and Stokal SR (sodium sulfosuccinamate) as comparison. Table 2 gives an overview of the compositions of the respective experiments.

All foaming experiments were conducted manually. For this purpose, polyurethane dispersion and surfactant were first placed in a 500 ml plastic cup and homogenized with a dissolver equipped with a disperser disc (diameter=6 cm) at 1000 rpm for 3 min. For foaming of the mixtures, the shear rate was then increased to 2000 rpm, ensuring that the disperser disc was always immersed into the dispersion to a sufficient degree that a proper vortex formed. At this speed, the mixtures were foamed to a volume of about 425 ml. The mixture was then sheared at 1000 rpm for a further 15 minutes. In this step, the disperser disc was immersed sufficiently deeply into the mixtures that no further air was introduced into the system, but the complete volume was still in motion.

TABLE 2
Overview of foam formulations:

| | #1 | | #2 | | #3 | |
|---|---|---|---|---|---|---|
| IMPRANIL ® DLU | 150 | g | 150 | g | 150 | g |

| | | | | |
|---|---|---|---|---|
| Surfactant 1 | 4 | g | — | — |

| | | | | |
|---|---|---|---|---|
| Surfactant 2 | — | 4 | g | — |

| | | | | |
|---|---|---|---|---|
| Stokal STA | — | — | 2 | g |
| Stokal SR | — | — | 2 | g |

| | | | |
|---|---|---|---|
| Wet foam viscosity [mPa s] | 7100 | 7400 | 4000 |

In all cases, fine homogeneous foams were obtained at the end of this foaming operation. It was noticeable that the foams #1 and #2 which had been produced with inventive surfactants 1 and 2 had a higher viscosity (see Table 2). The foams were applied to a siliconized polyester film with the aid of a film applicator (AB3220 from TQC) equipped with an applicator frame (film thickness=800 μm) and then dried at 60° C. for 5 min and at 120° C. for a further 5 min.

Compared to sample #3 thus obtained, the dried inventive samples #1 and #2 featured a more homogeneous macroscopic appearance and a more velvety feel. In electron microscopy studies, moreover, it was possible to ascertain a finer pore structure.

### Example 6: Migration Tests

To assess the surface migration of the surfactants according to the invention, imitation leather materials were produced by the method that follows. First of all, a topcoat coating was applied to a siliconized polyester film (layer thickness 100 μm). This was then dried at 100° C. for 3 minutes. Subsequently, a foam layer was coated onto the dried topcoat layer (layer thickness 800 μm) and dried at 60° C. for 5 minutes and at 120° C. for 5 minutes. In a last step, an aqueous adhesive layer (layer thickness 100 μm) was coated onto the dried foam layer, and then a textile carrier was laminated onto the still-moist adhesive layer. The finished laminate was dried again at 120° C. for 5 minutes and then detached from the polyester film.

All coating and drying operations were performed here with a Labcoater LTE-S from Mathis AG. Topcoat and adhesive layer were formulated here in accordance with the compositions listed in Table 3; the foam layers used were the foam formulations listed in Table 2, which were foamed by the method described in Example 5.

For assessment of surfactant migration, the imitation leather samples, after production, were placed into water at 100° C. for 30 minutes and then dried at room temperature overnight. After this treatment, the comparative sample produced from the Stokal STA/SR surfactants (foam formulation #3, Table 2) had distinctly visible white spots on the surface of the imitation leather, whereas this surface discoloration was not observed in the case of the samples produced with the surfactants according to the invention (foam formulation #1 and #2, Table 2).

TABLE 3
Topcoat and adhesive formulation for production
of imitation leather materials:

| | |
|---|---|
| Topcoat | Adhesive |

| | | | |
|---|---|---|---|
| CROMELASTIC ® PC 287 PRG | 100 | g | — |

| | | | |
|---|---|---|---|
| REGEL ® WX 151 | — | 100 | g |

| | | | | |
|---|---|---|---|---|
| ECO Pigment Black | 10 | g | 5 | g |
| TEGOWET ® 250 | 0.2 | g | 0.2 | g |
| REGEL ® TH 27 | 6 | g | 6 | g |
| ORTEGOL ® PV 301 | 7 | g | 5 | g |

### Example 7: Cosurfactant Compatibility

For assessment of cosurfactant compatibility, further foaming tests were conducted with the SYNTEGRA® YS:3000 PUD system. This contains 1-3% by weight of the anionic cosurfactant sodium dodecylbenzenesulfonate (CAS: 25155-30-0). The surfactants used in these experiments were the surfactant formulations 1-3 listed in Table 1. Table 4 gives an overview of the composition of the foam formulations.

TABLE 4
Overview of foam formulations:

| | | |
|---|---|---|
| #4 | #5 | #6 |

| | | | | | | |
|---|---|---|---|---|---|---|
| SYNTEGRA ® YS 3000 | 150 | g | 150 | g | 150 | g |

| | | | | |
|---|---|---|---|---|
| Surfactant 1 | 4 | g | — | — |

| | | | | |
|---|---|---|---|---|
| Surfactant 2 | — | 4 | g | — |

| | | | | |
|---|---|---|---|---|
| Surfactant 3 | — | — | 4 | g |

On the basis of these formulations, foam coatings were produced by the method described in Example 5. It was noticeable here that sample #6 produced with the noninventive surfactant 3 had a much coarser and less homogeneous foam structure. After the foam coating had dried, it was also possible to observe clear cracks in the foam structure, which is a pointer to inadequate stabilization of the foam. Samples #4 and #5 produced with the inventive surfactants, by contrast, again showed an extremely fine-cell and homogeneous foam structure. They were also free of drying cracks.

In addition, a further series of foaming experiments was conducted, in which the actually cosurfactant-free IMPRANIL® DLU system was deliberately additized with sodium dodecylbenzenesulfonate, a common cosurfactant for PUD stabilization as already described. In these experiments too, the surfactant formulations 1-3 listed in Table 1 were used. Table 5 gives an overview of the composition of the foam formulations.

TABLE 5
Overview of foam formulations:

| | #7 | | #8 | | #9 | |
|---|---|---|---|---|---|---|
| IMPRANIL ® DLU | 150 | g | 150 | g | 150 | g |
| Sodium dodecylbenzenesulfonate | 1.5 | g | 1.5 | g | 1.5 | g |

| | | | | |
|---|---|---|---|---|
| Surfactant 1 | 4 | g | — | — |

| | | | | |
|---|---|---|---|---|
| Surfactant 2 | — | 4 | g | — |

| | | | | |
|---|---|---|---|---|
| Surfactant 3 | — | — | 4 | g |

Here too, foam coatings were produced by the method described in Example 5. It was again noticeable here that sample #9 produced with noninventive surfactant 3 had drying cracks and a much coarser cell structure, whereas the two inventive samples #7 and #8 again showed a fine and homogeneous cell structure and were free of defects. Virtually no difference from the analogous, cosurfactant-free samples #1 and #2 (see Example 5) was observable here. These experiments thus demonstrate the distinct improvement in cosurfactant compatibility of the surfactants according to the invention.

1. An aqueous polymer dispersion additive comprising a two-tail long-chain anionic surfactants.

2. The aqueous polymer dispersion additive according to claim 1, wherein the two-tail long-chain anionic surfactant has an anionic hydrophilic head group and two identical or different long-chain hydrophobic hydrocarbyl radicals.

3. The aqueous polymer dispersion additive according to claim 1, wherein the two-tail long-chain anionic surfactant conforms to the general formula (I): 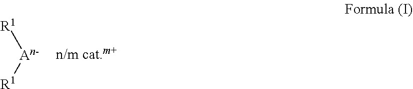
where the R¹ radicals are independently identical or different monovalent saturated or unsaturated, aliphatic or aromatic hydrocarbyl radicals having 12 to 40 carbon atoms, and where Aⁿ⁻ is the anionic head group of the surfactant that bears n negative charges, where n=1-3, and where cat.^{m+} is the cationic counterion that bears m positive charges, where m=1-10.

4. The aqueous polymer dispersion additive according to claim 1, wherein the R¹ radical derives from long-chain alcohols, carboxylic acids or alkylamines having at least 12 to 40 carbon atoms.

5. The aqueous polymer dispersion additive according to claim 1, wherein the R¹ radical derives from lauryl alcohol (1-dodecanol), myristyl alcohol (1-tetradecanol), cetyl alcohol (1-hexadecanol), margaryl alcohol (1-heptadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), melissyl alcohol (1-triacontanol), palmitoleyl alcohol (cis-9-hexadecen-1-ol), oleyl alcohol (cis-9-octadecen-1-ol) and/or elaidyl alcohol (trans-9-octadecen-1-ol) and mixtures of these substances, particular preference being given to cetyl alcohol and/or stearyl alcohol and to mixtures of these two substances, and/or derives from lauric acid (dodecanoic acid), myristic acid (tetradecanoic acid), palmitic acid (hexadecanoic acid), stearic acid (octadecanoic acid), arachic acid (eicosanoic acid), behenic acid (docosanoic acid), lignoceric acid (tetracosanoic acid), palmitoleic acid ((Z)-9-hexadecenoic acid), oleic acid ((Z)-9-hexadecenoic acid), elaidic acid ((E)-9-octadecenoic acid), cis-vaccenic acid ((Z)-11-octadecenic acid), linoleic acid ((9Z,12Z)-9,12-octadecadienoic acid), alpha-linolenic acid ((9Z,12Z,15Z)-9,12,15-octadecatrienoic acid), gamma-linolenic acid ((6Z,9Z,12Z)-6,9,12-octadecatrienoic acid), di-homo-gamma-linolenic acid ((8Z,11Z,14Z)-8,11,14-eicosatrienoic acid), arachidonic acid ((5Z,8Z,11Z,14Z)-5,8,11,14-eicosatetraenoic acid), erucic acid ((Z)-13-docosenoic acid), nervonic acid ((Z)-15-tetracosenoic acid), ricinoleic acid, hydroxystearic acid and/or undecenyloic acid, and also mixtures thereof, for example rapeseed oil acid, soya fatty acid, sunflower fatty acid, peanut fatty acid and/or tall oil fatty acid, particular preference being given to palmitic acid and/or stearic acid and to mixtures of these substances, and/or derives from laurylamine (1-dodecylamine), myristylamine (1-tetradecylamine), cetylamine (1-hexadecylamine), margarylamine (1-heptadecylamine), stearylamine (1-octadecylamine), arachidylamine (1-eicosylamine), behenylamine (1-docosylamine), lignocerylamine (1-tetracosylamine), cerylamine (1-hexacosylamine), montanylamine (1-octacosylamine), melissylamine (1-triacontylamine), palmitoleylamine (cis-9-hexadecenylamine), oleylamine (cis-9-octadecenylamine) and/or elaidylamine (trans-9-octadecenylamine) and mixtures of these substances, particular preference being given to cetylamine and/or stearylamine and to mixtures of these two substances.

6. The aqueous polymer dispersion additive according to claim 1, wherein the two-tail long-chain anionic surfactant conforms to the general formula (II):
R²-Aⁿ⁻n/m cat.^{m+} Formula (II)
where R² is a branched hydrocarbyl radical that in turn consists of two identical or different monovalent aliphatic or aromatic, saturated or unsaturated hydrocarbyl radicals each having 12 to 40 carbon atoms, and where Aⁿ⁻ is the anionic head group of the surfactant that bears n negative charges, where n=1-3, and where cat.^{m+} is the cationic counterion that bears m positive charges, where m=1-10.

7. The aqueous polymer dispersion additive according to claim 1, wherein the two-tail long-chain anionic surfactant conforms to the general formula (III) or (IV): 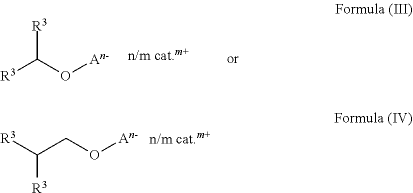
where the R³ radicals are independently identical or different monovalent aliphatic or aromatic, saturated or unsaturated hydrocarbyl radicals having 12 to 40 carbon atoms, and where Aⁿ⁻ is the anionic head group of the surfactant that bears n negative charges, where n=1-3, and where cat.^{m+} is the cationic counterion that bears m positive charges, where m=1-10.

8. The aqueous polymer dispersion additive according to claim 1, wherein the anionic head group Aⁿ⁻ is based on organic carboxylates or anionic sulfur or phosphorus compounds, where the head group in this connection has structural units selected from the group of the carboxylates, the phosphates, the phosphonates, the phosphinates, the sulfates and the sulfonates.

9. The aqueous polymer dispersion additive according to claim 1, wherein the two-tail long-chain anionic surfactant is selected from the group consisting of the dialkyl sulfosuccinates, dipalmityl sulfosuccinate, distearyl sulfosuccinate and/or stearyl palmityl sulfosuccinate, the group of the dialkyl sulfosuccinamates, dipalmityl sulfosuccinamate, distearyl sulfosuccinamate and/or stearyl palmityl sulfosuccinamate, the group of the dialkyl phosphates, dipalmityl phosphate, distearyl phosphate and/or stearyl palmityl phosphate, the group of the dialkyl esters of trifunctional or higher-functionality carboxylic acids, distearyl esters of trifunctional or higher-functionality carboxylic acids and stearyl palmityl esters of trifunctional or higher-functionality carboxylic acids, the group of the monoesters of phosphoric acid, sulfuric acid and dibasic or higher polybasic carboxylic acids with long-chain branched alcohols, the group of the monoalkyl sulfosuccinates based on long-chain branched alcohols, and mixtures of these substances.

10. The aqueous polymer dispersion additive according to claim 1, wherein the cation cat.^{m+} is a metal cation, and/or is an ammonium cation of a protonated amine compound.

11. The aqueous polymer dispersion additive according to claim 1, wherein the two-tail long-chain anionic surfactants are used in combination with at least one further cosurfactant as additive in aqueous polymer dispersions, where the cosurfactant preferably comprises fatty alcohols, fatty acid amides, ethylene oxide-propylene oxide block copolymers, betaines, amidopropyl betaines, amine oxides, quaternary ammonium surfactant, amphoacetates, ammonium and/or alkali metal salts of fatty acid, alkyl sulfates, alkyl ether sulfates, alkylsulfonates, alkylbenzenesulfonates, alkyl phosphates, alkyl sulfosuccinates, alkyl sulfosuccinamates, alkyl sarcosinates or else silicone-based cosurfactants and mixtures of these substances and alkyl sulfates having 12 to 40 carbon atoms, and to mixtures of these substances.

12. The aqueous polymer dispersion additive according to claim 1, wherein the aqueous polymer dispersions are selected from the group of aqueous polystyrene dispersions, polybutadiene dispersions, poly(meth)acrylate dispersions, polyvinyl ester dispersions and polyurethane dispersions, especially polyurethane dispersions, where the solids content of these dispersions is in the range of 20-70% by weight, based on the overall dispersion.

13. The aqueous polymer dispersion additive according to claim 1, wherein the concentration of the two-tail long-chain anionic surfactants based on the total weight of the aqueous polymer dispersion is in the range of 0.1-20% by weight.

14. An aqueous polyurethane dispersion, containing two-tail long-chain anionic surfactants, according to claim 1, wherein the solids content of this dispersion is in the range of 20-70% by weight, based on the overall dispersion, and wherein the concentration of the two-tail long-chain anionic surfactants based on the total weight of the aqueous polymer dispersion is in the range of 0.2-20% by weight.

15. A process for producing a porous polymer coating, using two-tail long-chain anionic surfactants as additives in aqueous polymer dispersions, according to claim 1, comprising the steps of:
a) providing a mixture comprising at least one aqueous polymer dispersion, at least one two-tail long-chain anionic surfactant according to the invention, and optionally further additives,
b) foaming the mixture to give a foam,
c) optionally adding at least one thickener to adjust the viscosity of the wet foam,
d) applying a coating of the foamed polymer dispersion, to a suitable carrier, and
e) drying the coating.

16. A porous polymer coating, obtainable by the use of two-tail long-chain anionic surfactants as additives in aqueous polymer dispersions, preferably aqueous polyurethane dispersions, in the production of such polymer coatings, obtained by a process according to claim 15.

17. A everyday article comprising a porous polymer coating according to claim 16, the everyday article being selected from the group consisting of shoes, insoles, bags, suitcases, small cases, clothing, automobile parts, preferably seat covers, coverings of door parts, dashboard parts, steering wheels and/or handles, and gearshift gaiters, desk pads, cushions or seating furniture, gap fillers in electronic devices, cushioning and damping materials in medical applications, and adhesive tapes.

18. The aqueous polymer dispersion additive according to claim 1, wherein the two-tail long-chain anionic surfactant conforms to the general formula (I): 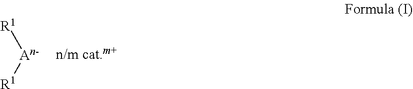
where the R¹ radicals are independently identical or different monovalent saturated or unsaturated, aliphatic or aromatic hydrocarbyl radicals having 16 to 24 carbon atoms, and where Aⁿ⁻ is the anionic head group of the surfactant that bears n negative charges, where n=1, and where cat.^{m+} is the cationic counterion that bears m positive charges, where m=1-2.

19. The aqueous polymer dispersion additive according to claim 1, wherein the R¹ radical derives from long-chain alcohols, carboxylic acids or alkylamines having at least 16 to 24 carbon atoms.

20. The aqueous polymer dispersion additive according to claim 1, wherein the two-tail long-chain anionic surfactant conforms to the general formula (II):
R²-Aⁿ⁻n/m cat.^{m+} Formula (II)
where R² is a branched hydrocarbyl radical that in turn consists of two identical or different monovalent aliphatic or aromatic, saturated or unsaturated hydrocarbyl radicals each having 16 to 24 carbon atoms, and wherein Aⁿ⁻ is the anionic head group of the surfactant that bears n negative charges, where n=1, and where cat.^{m+} is the cationic counterion that bears m positive charges, where m=1.
